(19) United States
(12) Patent Application Publication
(10) Pub. No.: US 2009/0056587 A1
(43) Pub. Date: Mar. 5, 2009

(54) Compound, Ink, Process And Use

(76) Inventor: Gavin Wright, Manchester (GB)

Correspondence Address: MORGAN LEWIS & BOCKIUS LLP, 1111 PENNSYLVANIA AVENUE NW, WASHINGTON, DC, 20004, US

(21) Appl. No.: 12/224,604

(22) PCT Filed: Apr. 27, 2007
(86) PCT No.: PCT/GB2007/001563

§ 371 Date: Sep. 2, 2008

(30) Foreign Application Priority Data
May 11, 2006 (GB) 0609309.0

(51) Int. Cl. C07D 403/02; C09D 11/02; B05D 5/00; B32B 3/10; B41J 2/175; G01D 11/00

(52) U.S. Cl. 106/31.48; 544/212; 427/256; 428/195.1; 347/86; 347/100

(57) ABSTRACT

A compound of Formula (1) or salt thereof:

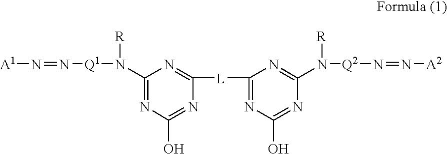

wherein:
A¹ and A² are each independently optionally substituted aryl;
Q¹ and Q² are each independently an optionally substituted arylene or polycyclic heteroarylene group each comprising a phenylene ring which is para connected to the —N═N— and the —NR— groups shown in Formula (1); and
each R is independently H or a substituent;
L is a linking group containing at least one group selected from sulfonic acid, phosphonic acid, carboxylic acid and sulfonamide groups. The compounds have good ozone and light fastness are especially suitable for use in ink jet printing inks.

This invention relates to compounds which are useful as colorants, to inks (especially ink jet printing inks) containing said compounds, to a process for printing said ink on a substrate and to the use of said compounds for preparing inks. Ink jet printing (hereinafter IJP) is a non-impact printing technique in which droplets of ink are ejected through a fine nozzle onto a substrate without bringing the nozzle into contact with the substrate.

There are many demanding performance requirements for colorants and inks used in IJP. For example, they desirably provide sharp, non-feathered images having good water-fastness, light fastness, ozone fastness and optical density. The inks are often required to dry quickly when applied to a substrate to prevent smudging, but they should not form a crust over the tip of an ink jet nozzle because this will tend to reduce print quality and in extreme cases may prevent the printer from printing. The inks should also be stable to storage over time without decomposing or forming a precipitate which could block the fine nozzle.

Azo containing colorants are known in the art.

JP1998279858 A and JP2000144003 A disclose yellow colorants for use in ink jet printing inks.

U.S. Pat. No. 5,328,995 discloses azo dyes useful in dyeing and printing fibre materials.

JP2005-00553, JP09-012950 and JP01-141965 disclose colorants useful in ink jet printing.

Chemical Abstracts 61:1974g-h/1975a-f discloses the preparation of metal complex dyes. However, further improvement in the properties of these colorants is still sought.

According to the present invention there is provided a compound of Formula (1) or a salt thereof:

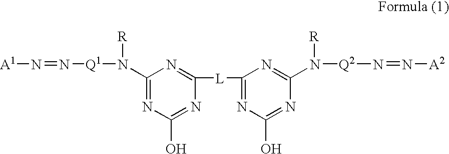

wherein:
A¹ and A² are each independently optionally substituted aryl;
Q¹ and Q² are each independently an optionally substituted arylene or polycyclic heteroarylene group each comprising a phenylene ring which is para connected to the —N═N— and the —NR— groups shown in Formula (1); and
each R is independently H or a substituent;
L is a linking group containing at least one group selected from sulfonic acid, phosphonic acid, carboxylic acid and sulfonamide groups.

A¹ and A² may each independently be either an optionally substituted polycyclic aryl group or monocyclic aryl group.

Preferred polycyclic aryl groups include naphthyl, anthracyl and pyrenyl groups. A preferred monocyclic aryl group is a phenyl group.

Preferably, A¹ and A are each independently naphthyl or phenyl.

Preferably, A¹ and A² are the same.

The optional substituents which may be present on A¹ and A² are preferably each independently selected from optionally substituted alkyl, alkoxy, amine, amide, ester, ketone and thioether groups and from halo, acid, hydroxy, nitro, cyano and —CF₃ groups.

Preferably, the optionally substituted alkyl group is a C₁₋₈ alkyl group, more preferably an optionally substituted C₁₋₄ alkyl group.

Preferably, the optionally substituted alkoxy group is a C₁₋₈-alkoxy group, more preferably an optionally substituted C₁₋₄-alkoxy group.

Preferably, the optionally substituted amine group is of the formula —NR¹R² wherein R¹ and R² are each independently H or optionally substituted alkyl, aryl or heteroaryl, or R¹ and R² together with the nitrogen atom to which they are attached form an optionally substituted 5- or 6-membered ring (e.g. a piperidine, pyrrolidone, pyridine, piperizine or morpholine ring).

Preferably, the optionally substituted amide group is of the formula —NHC(O)NR¹R², —C(O)NR¹R², —S(O)₂NR¹R² or —NHC(O)R³, wherein R³ is H or optionally substituted alkyl, aryl or heteroaryl and R¹ and R² are as hereinbefore defined.

Preferred optionally substituted ester groups are of the formula —C(O)OR⁴ or —S(O)₂OR⁴, wherein R⁴ is optionally substituted alkyl, aryl or heteroaryl.

Preferred optionally substituted ketone groups are of the formula —C(O)R⁴ wherein R⁴ is as hereinbefore defined.

Preferred optionally substituted thioether groups are of the formula —SR wherein R⁴ is as hereinbefore defined.

Optionally substituted alkyl, alkoxy, amine, amide, ester, ketone or thioether substituents may have one or more halo, amino, C₁₋₄-alkoxy, hydroxy and acid groups.

Preferred halo groups are Cl, F, Br and I.

Preferred acid groups are carboxylic acid, sulfonic acid and phosphonic acid groups.

Preferably, A¹ and A² each independently have from 1 to 4, more preferably from 1 to 3 groups selected from carboxylic acid, phosphonic acid and sulfonic acid groups. More preferably A¹ and A² each independently have from 1 to 3 sulfonic acid groups. In addition A¹ and A² may have one or more substituents other than carboxylic acid, phosphonic acid and sulfonic acid groups.

Preferably both Q¹ and Q² are arylene groups, more preferably Q¹ and Q² are the same arylene group.

The arylene group may be either polycyclic (e.g. naphthylene) or monocyclic (e.g. phenylene).

Preferably, Q¹ and Q² are each independently optionally substituted naphthylene or phenylene, more preferably both Q¹ and Q² are optionally substituted phenylene, in each case comprising a phenylene ring which is para connected to both the —N═N— and the —NR— groups shown in Formula (1).

Preferably, the optionally substituted polycyclic heteroarylene groups comprise a phenylene ring condensed with a 5- or 6-membered ring comprising one or more nitrogen, sulphur, oxygen or phosphorus atoms in the 5- or 6-membered ring (e.g. an indole group comprising a phenylene ring which is para connected to both the —N═N— and the —NR— groups in Formula (1)).

The optional substituents which may be present on Q¹ and Q² are preferably any of those described above for A¹ and A².

The optional substituents which may be present on Q¹ and Q² are preferably selected from C₁₋₄-alkoxy (especially methoxy and ethoxy), C₁₋₄-alkyl (especially methyl), —NHCONH₂, —NHSO₂—C₁₋₄-alkyl (especially —NHSO₂CH₃), carboxylic acid, sulfonic acid and phosphonic acid.

Preferably, Q¹ and Q² each have at least one substituent, more preferably Q¹ and Q² each have only one substituent.

Preferably, each R independently is H, optionally substituted alkyl or optionally substituted aryl. Preferred optionally substituted alkyl and optionally substituted aryl groups are as mentioned above for A¹ and A². More preferably each R independently is H or C₁₋₄-alkyl, especially H.

Preferably, the linking group L contains from 1 to 4, more preferably from 1 to 2 groups selected from sulfonic acid, phosphonic acid, carboxylic acid and sulfonamide groups.

Preferably, the linking group L is of formula -Z¹-M-Z²- wherein Z¹ and Z² are each independently of the formula NR⁵, S or O;

wherein R⁵ is H or optionally substituted alkyl, aryl or heteroaryl; and

M is an optionally substituted divalent organic group containing at least one group selected from sulfonic acid, phosphonic acid, carboxylic acid and sulfonamide groups.

The optional substituents on R⁵ may be any of those previously described for the groups represented by A¹ and A².

The optional substituents on M other than sulfonic acid, phosphonic acid, carboxylic acid and sulfonamide groups may be any of those previously described for the groups represented by A¹ and A².

Preferably, at least one of the groups represented by Z¹ and Z² is of the formula NR⁵ more preferably, both Z¹ and Z² are of the formula NH.

M is preferably an arylene group, more preferably a phenylene group. When M is an arylene group Z¹ and Z² are preferably attached to M in the 1 and 4 positions.

Preferably, the linking group L contains at least one group selected from sulfonic acid, phosphonic acid and carboxylic acid and groups.

Most preferably, the linking group L contains at least one carboxylic acid or sulfonic acid group.

In view of the forgoing the linking group L is preferably of Formula (2) or a salt thereof:

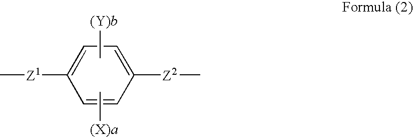

wherein:
Z¹ and Z² are each independently of the formula NR⁵, S or O wherein R⁵ is H or optionally substituted alkyl, aryl or heteroaryl;
each X independently is a group selected from sulfonic acid, phosphonic acid, carboxylic acid and sulfonamide groups;
a is from 1 to 4;
each Y is H or a substituent other than a sulfonic acid, phosphonic acid, carboxylic acid or sulfonamide group; and
b is 4-a.

When Y is a substituent this may be any of the substituents described for the groups A¹ and A² except sulfonic acid, phosphonic acid, carboxylic acid or sulfonamide groups.

Preferably, all the groups represented by Y are H. Preferably a is 1 or 2. Preferably, all groups represented by X are carboxylic acid groups.

Most preferably, the linking group L is of Formula (3) or a salt thereof:

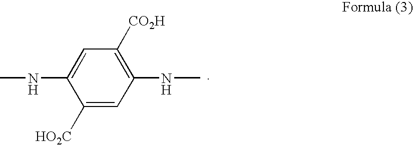

In a preferred embodiment the compound of Formula (1) is any of the compounds of Formulae (4) to (9) or salts thereof:

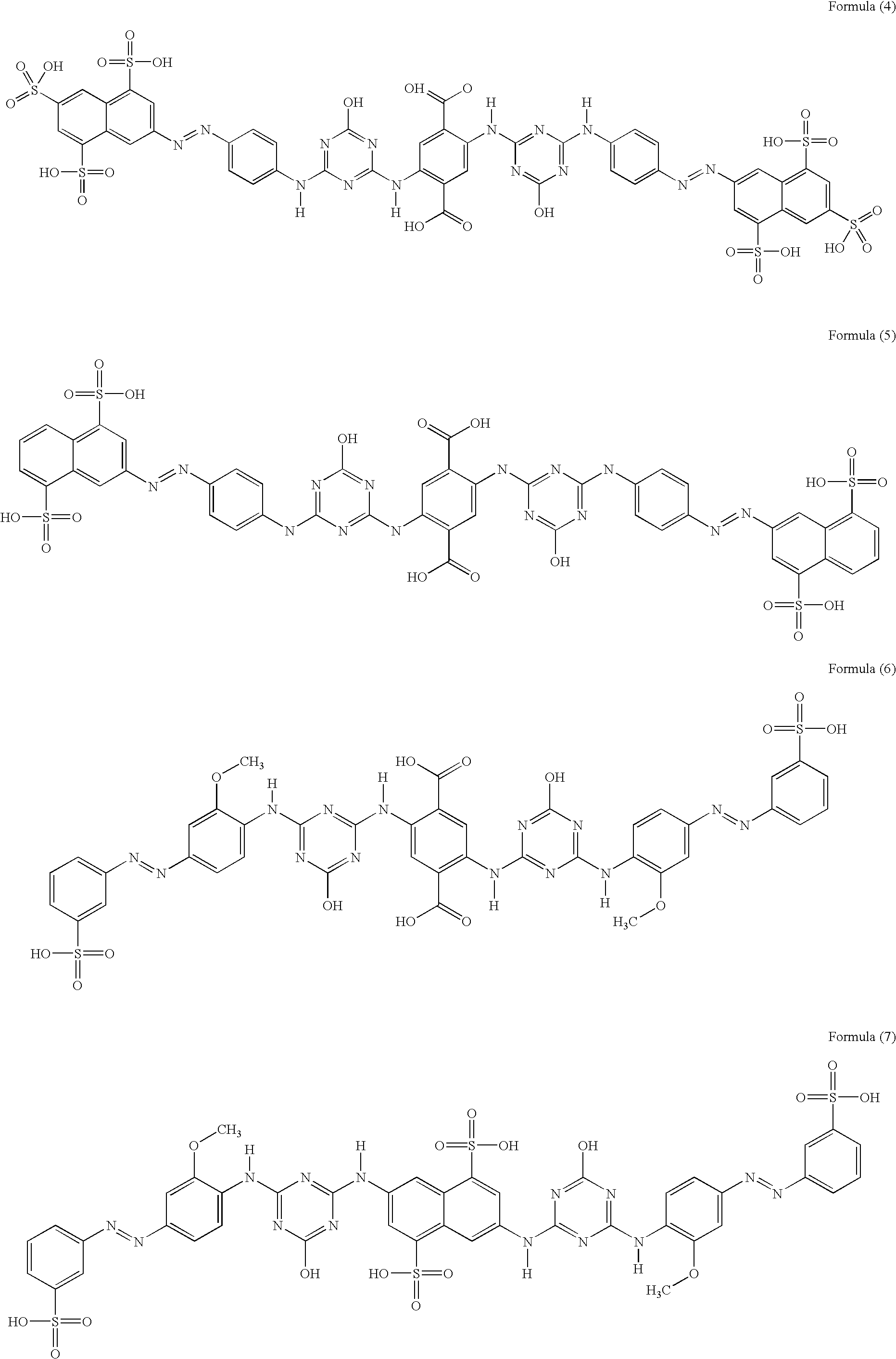

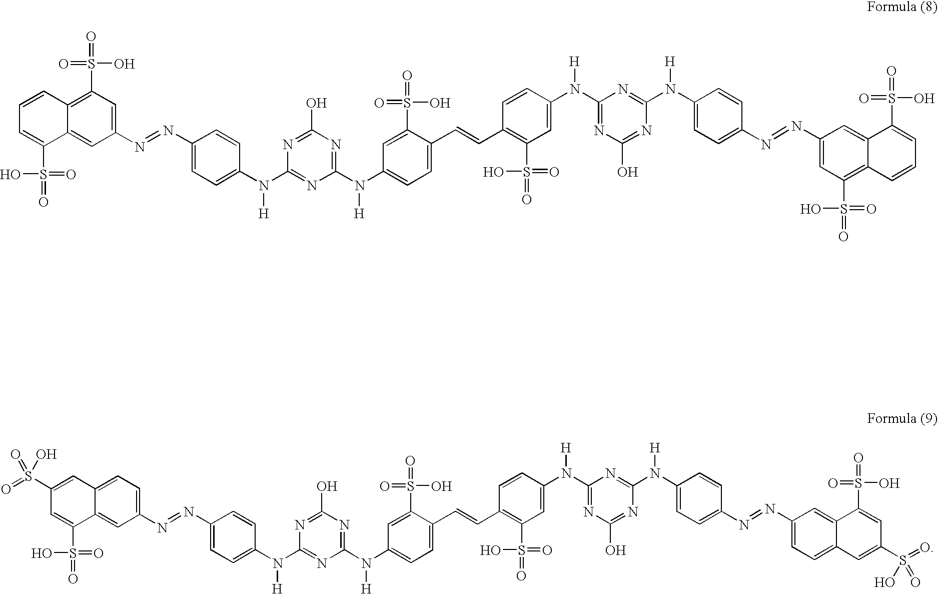

The compounds of Formula (1) exist in tautomeric forms other than those shown in this specification and such tautomers are included within the scope and claims of the present invention.

The compounds of Formula (1) may be in the protonated (free acid) or salt forms.

Accordingly, when the compound of Formula (1) contains groups such as sulphonic acid, phosphonic acid or carboxylic acid these may be in the form of the free acid (e.g. —SO₃H) or in the form of a salt (e.g. —SO₃Na).

Preferred salt forms are water-soluble, for example alkali metal salts (especially lithium, sodium, potassium), ammonium, substituted ammonium and mixed salts thereof.

Preferred ammonium and substituted ammonium salts are ammonium and alkyl or aryl substituted ammonium (e.g. ammonium, alkanolammonium, pyridinium, piperidinium and morpholinium).

It is especially preferred that the compounds of Formula (1) are in the form of a sodium, lithium, potassium or ammonium salt or a mixture thereof.

Preferably, the compound of Formula (1) or salt thereof has from 1 to 10 and especially from 2 to 8 groups selected from sulfonic acid, phosphonic acid and carboxylic acid groups.

Preferably, the compound of Formula (1) or salt thereof is a dye, more preferably a water-soluble dye.

Preferably, the compound of Formula (1) or salt thereof is yellow, orange or brown in colour. Preferably, inks containing about 5% by weight of the compound of Formula (1) or salt thereof are yellow in colour.

The compounds of the present invention are useful for preparing inks (especially IJP inks). These inks, when printed, exhibit particularly good ozone fastness, optical density and especially good light fastness.

Compounds of Formula (1) or salts thereof are preferably free from fibre reactive groups because such groups tend to reduce the long-term storage stability of inks. The term fibre reactive group is well understood in the art and is used for example in EP 0356014 A1. Fibre reactive groups are capable, under suitable conditions, of reacting with the hydroxy groups present in cellulosic fibres or with the amino groups present in natural fibres to form a covalent linkage between the fibre and the compound. Examples of fibre reactive groups which are preferably not present in the compounds of Formula (1) or salts thereof include: halo-1,3,5-triazinyl, halo-pyrimidyl, beta-halo-propionyl, beta-halo ethyl-sulphonyl, beta-sulfonic acid ethyl-sulfonyl, beta-halo ethylsulfamyl, chloroacetyl amino, beta-sulfonic acid ethyl sulphamoyl and vinyl sulphonyl groups.

### Preparation of Compounds of Formula (1)

The compounds of Formula (1) or salts thereof may be prepared by hydrolysis of the compound of Formula (10) or salt thereof:

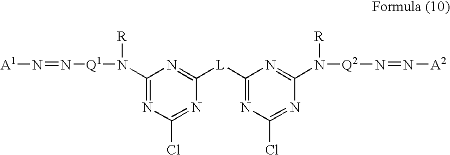

wherein A¹, A², Q¹, Q², R and L are as hereinbefore defined.

Preferably, the hydrolysis is performed in an alkaline solution containing sodium or lithium hydroxide at a temperature of 60 to 80° C. for a period of 4 to 10 hours.

The compound of Formula (10) or salt thereof is preferably prepared by reacting about 1 mole of a compound of formula HLH with 1 mole of a compound of Formula (11a) and 1 mole of a compound of (11b):

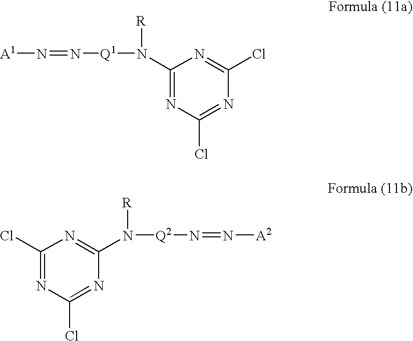

wherein A¹ A², Q¹, Q², R and L are as hereinbefore defined.

The reaction is preferably performed in aqueous solution at a temperature of from 20 to 40° C. and a pH of from 6 to 7.

For symmetrical compounds of Formula (1) or salt thereof HLH may be reacted with about two moles of a compound of Formula (11a) or salt thereof.

The compound of Formula (11a) or salt thereof is preferably prepared by reacting about 1 mole of cyanuric chloride with about 1 mole of a compound of Formula (12a) or salt thereof.

A¹-N═N-Q¹-NRH Formula (12a)

wherein A¹, Q¹ and R are as hereinbefore defined.

Compounds of Formula (11b) or salts thereof are prepared in exactly the same way as compounds of Formula (11a) or salts thereof except that the compound of Formula (12b) or salt thereof replaces the compound of Formula (12a) or salt thereof.

A²-N═N-Q2-NRH Formula (12b)

wherein A², Q² and R are as hereinbefore defined.

The compounds of Formula (12a) or salts thereof may be prepared by diazotising a compound of the Formula A¹-NH₂ to give a diazonium salt and coupling the resultant diazonium salt with a compound of Formula Q¹-NRH, wherein A¹, Q¹ and R are as hereinbefore defined.

The compounds of Formula (12b) or salts thereof may be prepared in an analogous manner to those of Formula (12a) or salts thereof by diazotising a compound of Formula A²-NH₂ to give a diazonium salt and coupling the resultant diazonium salt with a compound of Formula Q²-NRH, wherein A², Q² and R are as hereinbefore defined.

The diazotisation is preferably performed at a temperature below 20° C., more preferably at a temperature from 0° C. to 5° C. Preferably, the diazotisation is performed in a liquid comprising water, preferably having a very acidic pH (below 3). Mineral acids (e.g. HCl or H₂SO₄ or mixtures thereof) are typically used to achieve such an acidic pH.

The coupling reaction is preferably performed at a temperature of from 0 to 5° C., typically for a period of 1 to 6 hours. It is often desirable to add a buffer (e.g. sodium acetate) to adjust the pH to 4 to 5. The coupling reaction is preferably performed in a liquid comprising water. The coupling reaction is preferably continued for a further period of 16 hours at 25° C.

Preferably the compound of Formula HLH L is of Formula HZ¹-M-Z²H wherein

Z¹, Z² and M are as hereinbefore defined.

More preferably, the compound of Formula HLH is of Formula (13) or salt thereof:

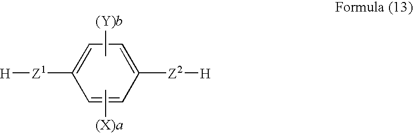

wherein Z¹, Z², X, a, Y and b are as hereinbefore defined.

It is especially preferred that the compound of Formula HLH is of Formula (14) or salt thereof:

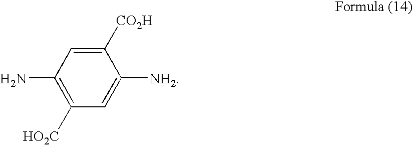

According to a second aspect of the present invention there is provided an ink comprising:
(a) at least one compound of Formula (1) or salt thereof according to the first aspect of the present invention; and
(b) a medium.

The medium is preferably a liquid medium or a low melting point solid medium.

More preferably, the ink according to the second aspect of the present invention comprises:
(a) from 0.01 to 30 parts of a compound of the Formula (1) or salt thereof; and
(b) from 70 to 99.99 parts of a liquid medium or a low melting point solid medium;
   wherein all parts are by weight and the number of parts of (a)+(b)=100.

The number of parts of component (a) is preferably from 0.1 to 20, more preferably from 0.5 to 15, and especially from 1 to 5 parts. The number of parts of component (b) is preferably from 99.9 to 80, more preferably from 99.5 to 85, especially from 99 to 95 parts.

Preferably, component (a) is completely dissolved in component (b). Preferably, component (a) has a solubility in component (b) at 20° C. of at least 10%. This allows the preparation of concentrates which may be used to prepare more dilute inks and reduces the chance of the compound of Formula (1) or salt thereof precipitating if evaporation of the liquid medium occurs during storage.

Preferred liquid media include water, a mixture of water and an organic solvent and an organic solvent free from water.

When the liquid medium comprises a mixture of water and an organic solvent, the weight ratio of water to organic solvent is preferably from 99:1 to 1:99, more preferably from 99:1 to 50:50 and especially from 95:5 to 80:20.

It is preferred that the organic solvent present in the mixture of water and organic solvent is a water-miscible organic solvent or a mixture of such solvents. Preferred water-miscible organic solvents include C₁₋₆-alkanols, preferably methanol, ethanol, n-propanol, isopropanol, n-butanol, sec-butanol, tert-butanol, n-pentanol, cyclopentanol and cyclohexanol; linear amides, preferably dimethylformamide or dimethylacetamide; ketones and ketone-alcohols, preferably acetone, methyl ether ketone, cyclohexanone and diacetone alcohol; water-miscible ethers, preferably tetrahydrofuran and dioxane; diols, preferably diols having from 2 to 12 carbon atoms, for example pentane-1,5-diol, ethylene glycol, propylene glycol, butylene glycol, pentylene glycol, hexylene glycol and thiodiglycol and oligo- and poly-alkyleneglycols, preferably diethylene glycol, triethylene glycol, polyethylene glycol and polypropylene glycol; triols, preferably glycerol and 1,2,6-hexanetriol; mono-C₁₋₄-alkyl ethers of diols, preferably mono-C₁₋₄-alkyl ethers of diols having 2 to 12 carbon atoms, especially 2-methoxyethanol, 2-(2-methoxyethoxy)ethanol, 2-(2-ethoxyethoxy)-ethanol, 2-[2-(2-methoxyethoxy)ethoxy]ethanol, 2-[2-(2-ethoxyethoxy)-ethoxy]-ethanol and ethyleneglycol monoallylether; cyclic amides, preferably 2-pyrrolidone, N-methyl-2-pyrrolidone, N-ethyl-2-pyrrolidone, caprolactam and 1,3-dimethylimidazolidone; cyclic esters, preferably caprolactone; sulphoxides, preferably dimethyl sulphoxide and sulpholane. Preferably the liquid medium comprises water and 2 or more, especially from 2 to 8, water-miscible organic solvents.

Especially preferred water-miscible organic solvents are cyclic amides, (especially 2-pyrrolidone, N-methyl-pyrrolidone and N-ethyl-pyrrolidone); diols, (especially 1,5-pentane diol, ethyleneglycol, thiodiglycol, diethyleneglycol and triethyleneglycol); and mono-C₁₋₄-alkyl and C₁₋₄-alkyl ethers of diols, more preferably mono-C₁₋₄-alkyl ethers of diols having 2 to 12 carbon atoms, especially 2-methoxy-2-ethoxy-2-ethoxyethanol.

A preferred liquid medium comprises:

(a) from 75 to 95 parts water; and
(b) from 25 to 5 parts in total of one or more organic solvents selected from diethylene glycol, 2-pyrrolidone, thiodiglycol, N-methylpyrrolidone, cyclohexanol, caprolactone, caprolactam and pentane-1,5-diol;
wherein the parts are by weight and the sum of the parts (a) and (b)=100.

Examples of further suitable ink media comprising a mixture of water and one or more organic solvents are described in U.S. Pat. No. 4,963,189, U.S. Pat. No. 4,703,113, U.S. Pat. No. 4,626,284 and EP 4,251,50A.

When the liquid medium comprises an organic solvent free from water, (i.e. less than 1% water by weight) the organic solvent preferably has a boiling point of from 30° to 200° C., more preferably of from 30° to 150° C., especially from 30 to 125° C. The organic solvent may be water-immiscible, water-miscible or a mixture of such solvents. Preferred water-miscible organic solvents are any of the hereinbefore described water-miscible organic solvents and mixtures thereof. Preferred water-immiscible solvents include, for example, aliphatic hydrocarbons; esters, preferably ethyl acetate; chlorinated hydrocarbons, preferably CH₂Cl₂; and ethers, preferably diethyl ether; and mixtures thereof.

When the liquid medium comprises a water-immiscible organic solvent, preferably a polar solvent is included because this enhances solubility of the compound of Formula (1) or salt thereof in the liquid medium. Examples of polar solvents include C₁₋₄-alcohols and ketones.

In view of the foregoing preferences it is especially preferred that where the liquid medium is an organic solvent free from water it comprises a ketone (especially methyl ethyl ketone) and/or an alcohol (especially a C₁₋₄-alkanol, more especially ethanol or propanol).

The organic solvent free from water may be a single organic solvent or a mixture of two or more organic solvents. It is preferred that when the medium is an organic solvent free from water it is a mixture of 2 to 5 different organic solvents. This allows a medium to be selected which gives good control over the drying characteristics and storage stability of the ink.

Ink media comprising an organic solvent free from water are particularly useful where fast drying times are required and particularly when printing onto hydrophobic and non-absorbent substrates, for example plastics, metal and glass.

Preferably, the low melting point solid is solid at 25° C. and melts at a temperature above 50° C. More preferably, the low melting point solid has a melting point in the range from 60° C. to 125° C.

Suitable media which melt in this temperature range include long chain fatty acids, sulfonamides or alcohols, preferably those having C₁₈₋₂₄ chains. The compound of Formula (1) or salt thereof may be dissolved in the low melting point solid or may be finely dispersed in it.

The ink according to the second aspect of the present invention may also contain additional components conventionally used in ink jet printing inks, for example viscosity and surface tension modifiers, corrosion inhibitors, biocides, kogation reducing additives and surfactants.

For inks in which the medium is a liquid medium the viscosity of the ink is preferably less than 50 mPa·s, more preferably less that 20 mPa·s and especially less than 5 mPa·s. The viscosity is preferably measured at a temperature of 25° C. Preferably, the viscosity is measured by means of a cone and plate rheometer at a shear rate corresponding to 100 rpm. Preferably, the ink is Newtonian in its viscosity behaviour (i.e. the viscosity is insensitive to shear rate).

Preferably, the ink has been filtered through a filter having an average pore size of less than 10 microns. More preferably the ink has been filtered through a filter having an average pore size of from 10 to 0.2, more preferably from 5 to 1 micron.

Preferably, the ink has a concentration of halide ions of less than 500 parts per million and more preferably less than 100 parts per million. It is especially preferred that the ink has less than 100, more preferably less than 50 parts per million in total of divalent and trivalent metals. Parts per million refer to parts by weight of the relevant ions or metals relative to the total weight of the ink. Any suitable means to remove chloride ions and/or di and trivalent metals may be employed, e.g. ion exchange and ultra-filtration. Preferably, the ink has been purified to a conductivity of less than 50 μS/cm. Such inks tend to work better with thermal ink jet printers.

According to a third aspect of the present invention there is provided a process for printing an image on a substrate comprising applying an ink containing a compound of Formula (1) or salt thereof according the first aspect of the present invention to the substrate.

Preferably, the ink is applied by means of an ink jet printer.

The ink used in this process is preferably as defined in the second aspect of the present invention.

According to a fourth aspect of the present invention there is provided a substrate (preferably paper, an overhead projector slide or a textile material) printed with an ink comprising a compound of Formula (1) or salt thereof according to the first aspect of the present invention. Preferably the ink is as defined in the second aspect of the present invention.

Preferred papers are plain or treated papers which may have an acid, alkaline or neutral character. Preferred substrates are those having a receptor layer for the ink. The receptor layer may be a porous or swellable layer.

According to a fifth aspect of the present invention there is provided an ink jet printer cartridge comprising a chamber and ink, wherein the ink is present in the chamber and the ink comprises a compound of Formula (1) or salt thereof according to the first aspect of the present invention. Preferably, the ink is as defined in the second aspect of the present invention.

According to a sixth aspect of the present invention there is provided the use of a compound of Formula (1) or salt thereof according to the first aspect of the present invention for preparing an ink (especially an ink jet printing ink) comprising said compound and a liquid medium wherein the liquid medium has a conductivity of less than 50 μS/cm. Such purified inks are especially useful in thermal ink jet printing where ppm levels of salts can kogate the ink jet printing heads and lead to reduced print quality over time.

The invention is further illustrated by the following Examples in which all parts and percentages are by weight unless otherwise stated.

### EXAMPLES

### Example 1

### Preparation of Dye (1)

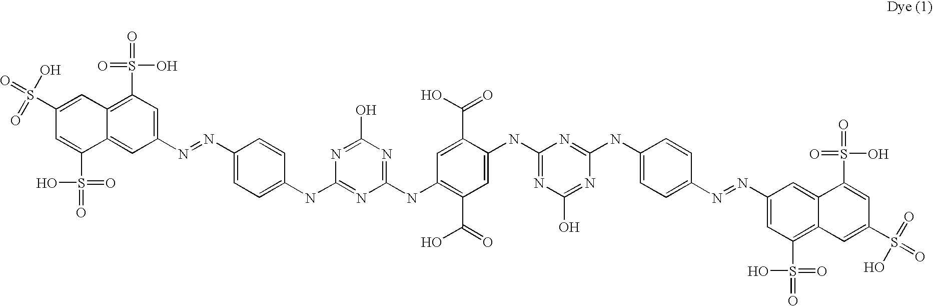

### Dye (1) was Prepared According to the Stages (A) to (C):

### Stage (a): Preparation of Intermediate (1a)

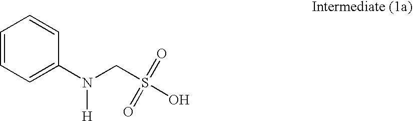

Aniline (511 g, 5.5 mol) was added slowly to a solution of formaldehyde/sodium-bisulfite addition compound (740 g, 5.5 mol) in water (1000 ml) at a temperature of 40° C. to form a reaction mixture.

The reaction mixture was stirred for 3 hours at a temperature of 50° C., then sodium chloride (100 g) was added and the reaction mixture was stirred for a further 3 hours whilst allowing the temperature of the reaction mixture to cool to a temperature of 25° C.

The product precipitated. The product was collected by filtration, washed with methylated spirit (3×300 ml) and dried to give 970 g of Intermediate (la) in the form of a white solid.

### Stage (b): Preparation of Intermediate (1b)

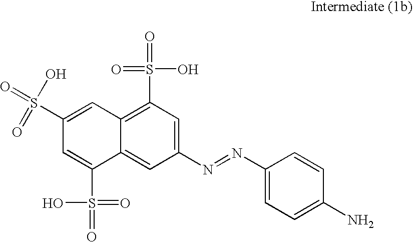

7-aminonaphthalene-1,3,5-trisulfonic acid (76.6 g, 0.2 mol) was dissolved in water (800 ml) and this was adjusted to a pH of 7 by the addition of 2N sodium hydroxide solution, then sodium nitrite (13.8 g, 0.2 mol) was added to form a solution.

The above solution was added dropwise to a mixture of concentrated sulphuric acid (60 g) and water (150 ml) at a temperature of from 0 to 5° C. to form a reaction mixture.

The reaction mixture was stirred for a period of 2 hours at a temperature of from 0 to 5° C. Intermediate (la) from stage (a) (37.4 g, 0.2 mol) was added in portions over a period of 1 hour to the reaction mixture at a temperature of from 0 to 5° C., the pH was adjusted to 4-5 by the addition of sodium acetate and the reaction mixture was stirred for a further period of 4 hours still at a temperature of from 0 to 5° C. Sodium hydroxide (100 g) was added to the reaction mixture which was then stirred at a temperature of 80° C. for a further 6 hours.

The reaction mixture was allowed to cool to 25° C. and the product was precipitated by the addition of sodium chloride (200 g). The product was collected by filtration and dried to give 58 g of Intermediate (1b) in the form of an orange solid.

### Stage (c) Preparation of Dye (1):

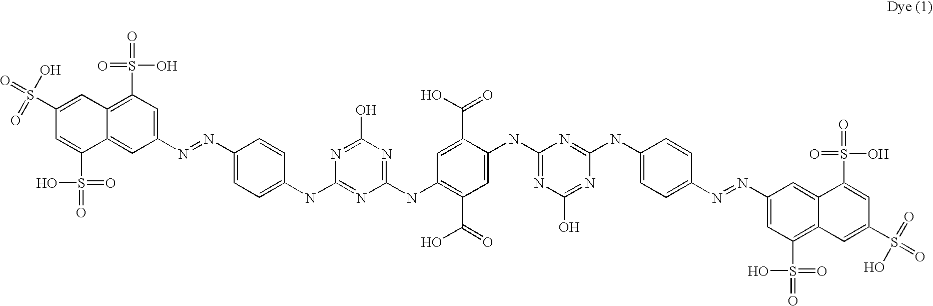

A solution of cyanuric chloride (9.2 g, 0.05 mol) in acetone (100 ml) was added to a mixture of ice/water (300 g) to form a cyanuric chloride suspension.

Intermediate (1b) from stage (b) (24.4 g, 0.05 mol) was dissolved in water (200 ml), adjusted to pH 7 by the addition of 2N lithium hydroxide solution and then added to the above described cyanuric chloride suspension at a temperature of from 0 to 5° C. to form a reaction mixture. The pH of the reaction mixture was maintained at 5-6.5 using 2N lithium hydroxide solution for a period of 1 hour.

2,5-diaminoterephthalic acid (4.9 g, 0.025 mol) was dissolved in water (200 ml), which was then adjusted to pH 7 by the addition of 2N lithium hydroxide solution to form a solution. The solution was then added to the reaction mixture. The reaction mixture was stirred at a temperature of 35° C. and a pH of 7-8 (using 2N lithium hydroxide solution) for a period of 18 hours. The reaction mixture was allowed to cool to 25° C. and the resultant precipitate was collected by filtration, washed with acetone (200 ml) and dried to give a solid.

The above solid was dissolved in a solution of LiOH (50 g) in water (500 ml) and the resultant solution was stirred at a temperature of 75° C. for a period of 3 hours. The product was precipitated by the addition of lithium chloride (100 g) and collected by filtration. The product was dissolved in water (300 ml) and purified by dialysis in membrane tubing to conductivity of less than 50 μS/cm. After evaporating the water at a temperature of 60° C. Dye (1) was obtained (9.5 g) in the form of an orange solid. Dye (1) is obtained in the form of the lithium salt.

### Examples 2 to 6

Dyes (2) to (6) were prepared in exactly the same way as described above for example 1 except that:
(i) 7-aminonaphthalene-1,3,5-trisulfonic acid in stage (b) of Example 1 was replaced by the compounds shown in column A of Table 1
(ii) the product from stage (a) of Example 1 was replaced by the compounds shown in column B of Table 1 which is prepared in stage (i); and
(iii) 2,5-diaminoterephthalic acid in Example 1 was replaced with the compounds shown in column C of Table 1.

TABLE 1

| Ex | Dye | A | B | C |
|---|---|---|---|---|
| 2 | Dye(2) | 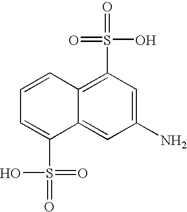 | 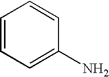 | 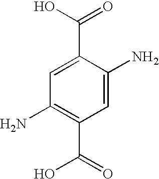 |
| 3 | Dye(3) | 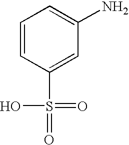 | 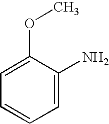 | 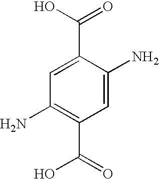 |
| 4 | Dye(4) | 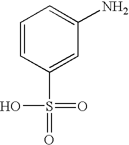 | 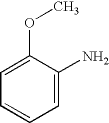 | 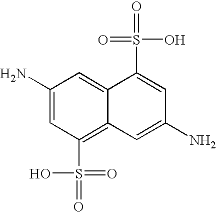 |
| 5 | Dye(5) | 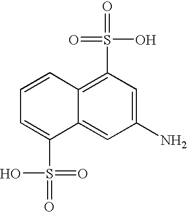 | 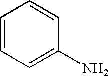 | 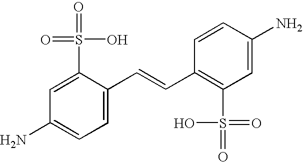 |
| 6 | Dye(6) | 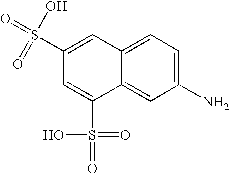 | 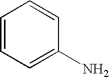 | 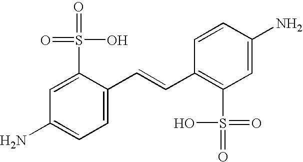 |

The final structures of Dyes (1) to (6) were those of Compounds of Formulae (4) to (9) respectively in the form of the lithium salt. The Dyes (1) to (6) may readily be converted into other salts such as sodium, potassium or ammonium by any conventional means (for example by ion exchange).

Dyes (1) to (6) may be used to prepare ink jet printing inks which demonstrate particularly good ozone and light fastness.

### Comparative Example 1

Comparative Dye (1) was prepared in exactly the same way as described above for example 1 except that:
(i) 7-aminonaphthalene-1,3,5-trisulfonic acid in stage (b) of Example 1 was replaced by the compound shown in column A of Table 2
(ii) the product from stage (a) of Example 1 was replaced by the compound shown in column B of Table 2 which is prepared in stage (i); and
(iii) 2,5-diaminoterephthalic acid in Example 1 was replaced with the compound shown in column C of Table 2.

TABLE 2

| C. Ex | Dye | A | B | C |
|---|---|---|---|---|
| 1 | C. Dye(1) | 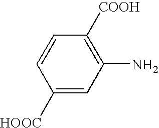 | 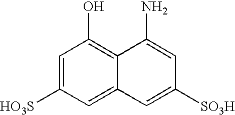 | 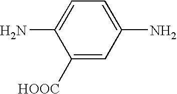 |

### Comparative Dye (1) had the Formula (15):

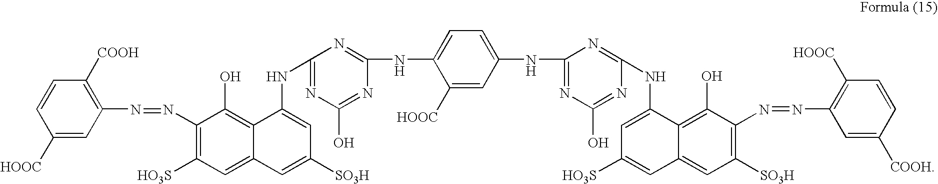

The compound of Formula (15) is a comparative compound because the naphthylene rings are not para coupled. Comparative Dye (1) is the same compound as Structure D in JP 09-012950.

### Inks

Inks (1) to (4) and Comparative Ink (1) were prepared by mixing the components as indicated in Table 3.

TABLE 3

| | | | | | Comparative Ink |
|---|---|---|---|---|---|
| Components | Ink (1) | Ink (2) | Ink (3) | Ink (4) | (1) |
| Thiodiglycol | 5 | 5 | 5 | 5 | 5 |
| 2-Pyrrolidone | 5 | 5 | 5 | 5 | 5 |
| Surfynol ™ | 1 | 1 | 1 | 1 | 1 |
| 465 | | | | | |
| Water | 86 | 86 | 86 | 86 | 86 |
| Dye (2) | 3 | | | | |
| Dye (1) | | 3 | | | |
| Dye (6) | | | 3 | | |
| Dye (5) | | | | 3 | |
| Comparative | | | | | 3 |
| Dye (1) | | | | | |

Inks (1) to (4) and Comparative Ink (1) were adjusted to a pH of 9.

Surfynol™ 465 is a surfactant available from Air Products.

### Printing

Inks (1) to (4) and Comparative Ink (1) were printed onto Canon PR 101 paper using a Canon i965 ink jet printer to produce two sets of Prints (1) to (4) and Comparative Print (1).

### Testing

One set of Prints (1) to (4) and Comparative Print (1) were subjected to ozone exposure in the dark for 48 hours at a concentration of 5 ppm ozone using a cabinet from Hampden Test Equipment.

The second set of prints (1) to (4) and Comparative Print (1) were exposed to light in an Atlas Ci35 Weatherometer™ for 100 hours.

The Reflectance optical density of the prints was measured using a Gretag Macbeth Spectrolino Spectrodensitometer before and after ozone or light exposure.

### Results

The % loss in the reflectance optical density after ozone or light exposure was as shown in Table 4.

TABLE 4

| | % loss in Reflectance | % loss in Reflectance |
|---|---|---|
| | optical density after | optical density after |
| Print | ozone exposure | light exposure |
| Print (1) | 38 | 15 |
| Print (2) | 33 | 12 |
| Print (3) | 38 | 16 |
| Print (4) | 39 | 17 |
| Comparative Print (1) | 70 | 52 |

A lower value of % loss in Reflectance optical density indicates better fastness. Table 4 clearly shows that the ozone fastness and light fastness of the prints derived from inks containing compounds according to the present invention are much superior to prints derived from inks containing compounds known in the art.

### Further Inks

The further inks described in Tables 1 and 11 may be prepared wherein the Dye described in the first column is the Dye made in the above Example of the same number. Numbers quoted in the second column onwards refer to the number of parts of the relevant ingredient and all parts are by weight. The inks may be applied to paper by thermal or piezo ink jet printing.

The following abbreviations are used in Table I and II:

PG=propylene glycol

DEG=diethylene glycol

NMP=N-methylpyrrolidone

DMK=dimethylketone

IPA=isopropanol

MeOH=methanol

2P=2-pyrrolidone

MIBK=methylisobutyl ketone

P12 propane-1,2-diol

BDL=butane-2,3-diol

CET=cetyl ammonium bromide

PHO=Na₂HPO₄ and

TBT=tertiary butanol

TDG=thiodiglycol

TABLE I

| | Dye | | | | | | | Na | | | | |
|---|---|---|---|---|---|---|---|---|---|---|---|---|
| Dye | Content | Water | PG | DEG | NMP | DMK | NaOH | Stearate | IPA | MEOH | 2P | MIBK |
| 1 | 2.0 | 80 | 5 | | 6 | 4 | | | | | 5 | |
| 2 | 3.0 | 90 | | 5 | 5 | | 0.2 | | | | | |
| 3 | 10.0 | 85 | 3 | | 3 | 3 | | | | 5 | 1 | |
| 4 | 2.1 | 91 | | 8 | | | | | | | | 1 |
| 5 | 3.1 | 86 | 5 | | | | | 0.2 | 4 | | | 5 |
| 6 | 1.1 | 81 | | | 9 | | 0.5 | 0.5 | | | 9 | |
| 1 | 2.5 | 60 | 4 | 15 | 3 | 3 | | | 6 | 10 | 5 | 4 |
| 2 | 5 | 65 | | 20 | | | | | 10 | | | |
| 3 | 2.4 | 75 | 5 | 4 | | 5 | | | | 6 | | 5 |
| 4 | 4.1 | 80 | 3 | 5 | 2 | 10 | | 0.3 | | | | |
| 5 | 3.2 | 65 | | 5 | 4 | 6 | | | 5 | 4 | 6 | 5 |
| 6 | 5.1 | 96 | | | | | | | | 4 | | |
| 1 | 10.8 | 90 | 5 | | | | | | 5 | | | |
| 2 | 10.0 | 80 | 2 | 6 | 2 | 5 | | | 1 | | 4 | |
| 3 | 1.8 | 80 | | 5 | | | | | | | 15 | |
| 4 | 2.6 | 84 | | | 11 | | | | | | 5 | |
| 5 | 3.3 | 80 | 2 | | | 10 | | | | 2 | | 6 |
| 6 | 12.0 | 90 | | | | 7 | 0.3 | | 3 | | | |
| 1 | 5.4 | 69 | 2 | 20 | 2 | 1 | | | | | 3 | 3 |
| 2 | 6.0 | 91 | | | 4 | | | | | | 5 | |

TABLE II

| | Dye | | | | | | | | | | | |
|---|---|---|---|---|---|---|---|---|---|---|---|---|
| Dye | Content | Water | PG | DEG | NMP | CET | TBT | TDG | BDL | PHO | 2P | PI2 |
| 3 | 3.0 | 80 | 15 | | | 0.2 | | | | | 5 | |
| 4 | 9.0 | 90 | | 5 | | | | | | 1.2 | | 5 |
| 5 | 1.5 | 85 | 5 | 5 | | 0.15 | 5.0 | 0.2 | | | | |
| 6 | 2.5 | 90 | | 6 | 4 | | | | | 0.12 | | |
| 1 | 3.1 | 82 | 4 | 8 | | 0.3 | | | | | | 6 |
| 2 | 0.9 | 85 | | 10 | | | | | 5 | 0.2 | | |
| 3 | 8.0 | 90 | | 5 | 5 | | | 0.3 | | | | |
| 4 | 4.0 | 70 | | 10 | 4 | | | | 1 | | 4 | 11 |
| 5 | 2.2 | 75 | 4 | 10 | 3 | | | | 2 | | 6 | |
| 6 | 10.0 | 91 | | | 6 | | | | | | 3 | |
| 1 | 9.0 | 76 | | 9 | 7 | | 3.0 | | | 0.95 | 5 | |
| 2 | 5.0 | 78 | 5 | 11 | | | | | | | 6 | |
| 3 | 5.4 | 86 | | | 7 | | | | | | 7 | |
| 4 | 2.1 | 70 | 5 | 5 | 5 | 0.1 | 0.2 | 0.1 | 5 | 0.1 | 5 | |
| 5 | 2.0 | 90 | | 10 | | | | | | | | |
| 1 | 2 | 88 | | | | | | 10 | | | | |
| 2 | 5 | 78 | | | 5 | | | 12 | | | 5 | |
| 1 | 8 | 70 | 2 | | 8 | | | 15 | | | 5 | |
| 1 | 10 | 80 | | | | | | 8 | | | 12 | |
| 1 | 10 | 80 | | 10 | | | | | | | | |

1. A compound of Formula (1) or salt thereof: 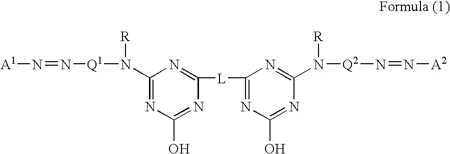
wherein:
A¹ and A² are each independently optionally substituted aryl;
Q¹ and Q² are each independently an optionally substituted arylene or polycyclic heteroarylene group each comprising a phenylene ring which is para connected to the —N═N— and the —NR— groups shown in Formula (1); and
each R is independently H or a substituent;
L is a linking group containing at least one group selected from sulfonic acid, phosphonic acid, carboxylic acid and sulfonamide groups.

2. A compound or salt thereof according to claim 1 wherein:
L is of Formula -Z¹-M-Z²-;
Z¹ and Z² are each independently of the formula NR⁵, S or O;
R⁵ is H or optionally substituted alkyl, aryl or heteroaryl; and
M is an optionally substituted divalent organic group containing at least one group selected from sulfonic acid, phosphonic acid, carboxylic acid and sulfonamide groups.

3. A compound or salt thereof according to claim 2 wherein at least one of the groups represented by Z¹ and Z² is of the formula NR⁵.

4. A compound or salt thereof according to claim 1 wherein the linking group L contains at least one group selected from sulfonic acid, phosphonic acid and carboxylic acid groups.

5. A compound or salt thereof according to claim 1 wherein the linking group L contains at least one carboxylic acid or sulfonic acid group.

6. A compound or salt thereof according to claim 1 wherein the linking group L is of Formula (2): 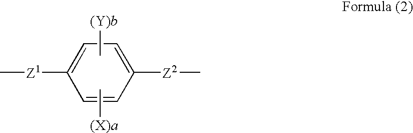
wherein:
Z¹ and Z² are each independently of the formula NR⁵, S or O wherein R⁵ is H or optionally substituted alkyl, aryl or heteroaryl;
each X independently is selected from sulfonic acid, phosphonic acid, carboxylic acid and sulfonamide groups;
a is from 1 to 4;
each Y is H or a substituent other than a sulfonic acid, phosphonic acid, carboxylic acid or sulfonamide group; and
b is 4-a.

7. A compound or salt thereof according to claim 1 wherein the linking group L is of Formula (3):

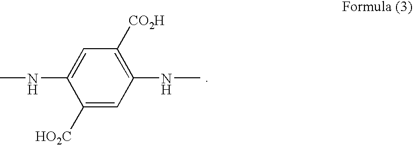

8. A compound or salt thereof according to claim 1 which is a water-soluble dye.

9. A compound or salt thereof according to claim 1 having from 1 to 10 groups selected from sulfonic acid, phosphonic acid and carboxylic acid groups.

10. A salt of a compound according to claim 1 which is in the form of an alkali metal salt, ammonium or substituted ammonium salt or a mixture thereof.

11. An ink comprising:
(a) at least one compound of Formula (1) or salt thereof according to claim 1; and
(b) a medium.

12. An ink according to claim 11 wherein the medium is a liquid medium.

13. A process for printing an image on a substrate comprising applying an ink containing a compound according to claim 1 to the substrate.

14. A process according to claim 13 wherein the ink is applied by means of a ink jet printer.

15. A substrate printed with an ink comprising a compound of Formula (1) or salt thereof according to claim 1.

16. An ink jet printer cartridge comprising a chamber and ink, wherein the ink is present in the chamber and the ink comprises a compound of Formula (1) or salt thereof according to claim 1.

17. An ink comprising a compound of Formula (1) or salt thereof according to claim 1 for preparing an ink comprising the compound and a liquid medium wherein the liquid medium has a conductivity of less than 50 μS/cm.
